(19) United States
(12) Patent Application Publication
(10) Pub. No.: US 2012/0253308 A1
(43) Pub. Date: Oct. 4, 2012

(54) ARTICLE INCLUDING VISUAL SIGNAL FOR COMMUNICATION OF A FUNCTIONAL BENEFIT

(76) Inventors: Joanna Misiti, Hermosa Beach, CA (US); Neil Alexander Sadler, Weehawken, NJ (US); Aimee Sealfon, Brooklyn, NY (US); Dana Snider, Brooklyn, NY (US)

(21) Appl. No.: 13/079,159

(22) Filed: Apr. 4, 2011

(51) Int. Cl. A61F 13/84

(52) U.S. Cl. 604/385.01

(57) ABSTRACT

The present invention provides an absorbent article including a longitudinally extending a top surface, a cover layer, a barrier layer, an absorbent core arranged between the cover layer and the barrier layer, a printed portion, a plurality of non-printed portions, wherein the printed portion and the plurality of non-printed portions are arranged to create a perception of a functional benefit by a user looking upon the top surface of the absorbent article.

### FIELD OF INVENTION

Absorbent articles according to the present invention are provided with a plurality of non-printed portions and a printed portion, the plurality of non-printed portions and the printed portion cooperating to visually communicate a functional benefit to the user.

### BACKGROUND OF THE INVENTION

Due to improvements in materials that constitute disposable sanitary absorbent articles (such as sanitary napkins, pantiliners and the like), and improvements in the construction of such articles, commercially available disposable sanitary absorbent articles can provide excellent fluid handling properties. Further, it is known in the prior art to provide disposable absorbent articles with other functional properties. For example, it is known to provide the cover layer of such articles with a skin soothing agent to thereby provide a skin soothing benefit to such articles. However, much of the technology that provides enhanced fluid handling properties, or other functional properties to the article, is hidden from the consumer. Consequently, these enhanced functional properties are not readily communicated to the user, visually or otherwise.

In view of the above, a unique absorbent article structure is disclosed herein wherein the article includes a plurality of non-printed portions and a printed portion, the plurality of non-printed portions and printed portion cooperating to visually communicate a functional benefit to the user.

### SUMMARY OF THE INVENTION

In view of the foregoing the present invention provides an absorbent article including a longitudinally extending axis, a transversely extending axis, a top surface, a cover layer, a barrier layer, an absorbent core arranged between the cover layer and the barrier layer, a printed portion, a plurality of non-printed portions, wherein the printed portion and the plurality of non-printed portions are arranged to create a perception of a functional benefit by a user looking upon the top surface of the absorbent article.

### BRIEF DESCRIPTION OF THE DRAWINGS

Examples of embodiments of the present invention will now be described with reference to the drawings, in which:

FIG. 1 is a top perspective view of an absorbent article according to a first embodiment of the present invention;

FIG. 2 is an exploded view of the absorbent article shown in FIG. 1;

FIG. 3 is a top perspective view of an absorbent article according to a second embodiment of the present invention; and

FIG. 4 is an exploded view of the absorbent article shown in FIG. 3.

### DETAILED DESCRIPTION OF THE INVENTION

The present invention generally relates to disposable sanitary absorbent articles such as sanitary napkins, pantiliners, absorbent products for incontinence, baby diapers, and other disposable absorbent articles adapted to absorb body exudates. The term “disposable” as used herein refers to absorbent articles that are not intended to be laundered or otherwise reused as an absorbent article. Although the invention will be described herein with reference to a sanitary napkin, the invention may be utilized with other disposable sanitary absorbent articles.

Absorbent articles according to the present invention are provided with a plurality of non-printed portions and a printed portion viewable from a top surface of the absorbent article. The plurality of non-printed portions and the printed portion cooperate to visually communicate a functional benefit to the user. The “functional benefit” communicated by embodiments of the present invention include at least one of breathability of the absorbent article, dryness, temperature control (e.g. a product that feels cool during use), body fit (i.e. the body conforming nature of the product), comfort, and combinations thereof. In this way the visual signal of the article visually communicates to the user that the absorbent article includes technology intended to deliver these attributes during use. The printed portion of the article should have a color that is readily visible and distinguishable from the color of the non-printed portions by a user having 20/20 vision viewing the top surface of the article at a distance of twelve inches from the top surface.

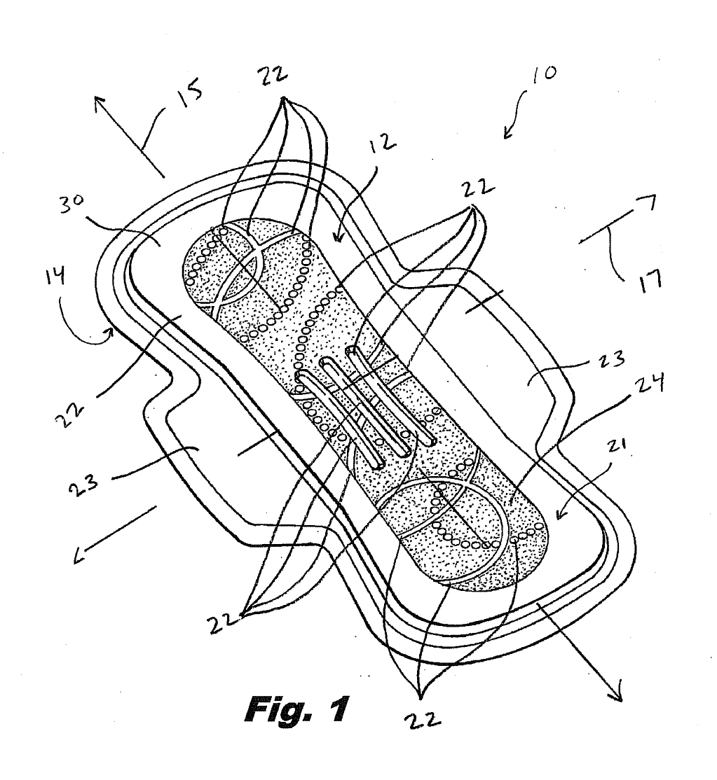

FIG. 1 provides a perspective view of a sanitary napkin 10 in accordance with a first embodiment of the present invention. The sanitary napkin 10 includes a top surface 12 and a bottom surface 14. The sanitary napkin 10 further includes a longitudinally extending centerline 15 and a transversely extending centerline 17.

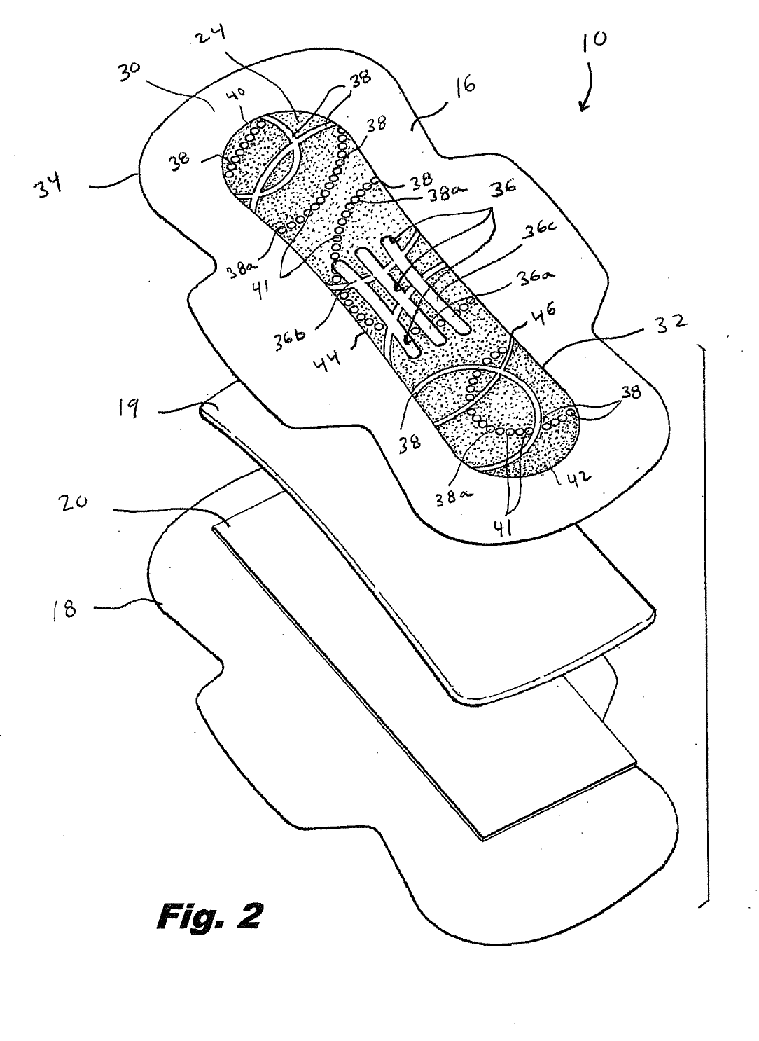

As shown in FIG. 2, the sanitary napkin 10 includes a liquid permeable cover layer 16, a liquid impermeable barrier layer 18, and an absorbent core 20 arranged between the cover layer 16 and the barrier layer 18. The sanitary napkin 10 may optionally include a transfer layer 19 arranged between the cover layer 16 and the core 20. As shown in FIG. 1, the sanitary napkin 10 includes a main body 21, which includes the primary absorbent portions of the napkin, and a pair of wings 23 that extend outwardly from the main body 21.

As shown in FIG. 1, the sanitary napkin 10 according to the first embodiment of the present invention generally includes a plurality of non-printed portions 22 and a printed portion 24. Preferably each of the plurality of non-printed portions 22 has a color that is the same color as all of the other non-printed portions 22. The non-printed portions 22 and the printed portion 24 are viewable from the top surface 12 of the sanitary napkin 10. The non-printed portions 22 and printed portion 24 cooperate to create a perception of at least one of breathability of the absorbent article, dryness, temperature control (e.g. a product that feels cool during use), body fit (i.e. the body conforming nature of the product), comfort, and combinations thereof, by a user looking upon the top surface 12 of the sanitary napkin 10.

In the embodiment shown in FIG. 1, the printed portion 24 is centrally arranged on the main body 21 and extends substantially in a longitudinal direction of the absorbent article. In the specific embodiment shown in FIG. 1, the printed portion 24 is arranged such that it is symmetrical with respect to the longitudinally extending axis 15 and the transversely extending axis 17. As shown in FIG. 2, the printed portion 24 includes a first substantially transversely extending peripheral edge 40, a second opposed substantially transversely extending peripheral edge 42, a first longitudinally extending peripheral edge 44 and an opposed second longitudinally extending peripheral edge 46.

In the specific embodiment shown in FIG. 1, the printed portion 24 is substantially peanut shaped and extends over a surface area of between about 24% and about 30% of a total surface area of the top surface 12 of the main body 21 (i.e. excluding the area of the top surface 12 of the wings 23). Preferably, the printed portion 24 extends over an area of between about 5203 mm² and about 6302 mm². Preferably, the printed portion 24 is configured such that the entirety of the printed portion 24 has a single color.

As shown in FIG. 2, the plurality of non-printed portions 22 includes a first non-printed portion 30 that completely surrounds the printed portion 24. In the embodiment shown in FIGS. 1 and 2, the first non-printed portion 30 extends from a peripheral boundary 32 of the printed portion 24 (See FIG. 2) to a peripheral edge 34 of the main body 21.

As shown in FIG. 2, the plurality of non-printed portions 22 further includes a plurality of substantially longitudinally extending non-printed portions 36 that are substantially centrally arranged within the printed portion 24. The longitudinally extending non-printed portions 36 include a first central non-printed portion 36a that is symmetrically arranged with respect to the longitudinally extending axis 15 and the transversely extending axis 17. The plurality of substantially longitudinally extending non-printed portions 36 further include a second 36b and third 36c non-printed portion that are arranged in spaced relationship to the first central non-printed portion 36a and are located on opposite sides of the longitudinally extending axis 15. The second 36b and third 36b non-printed portions are symmetrically arranged with respect to the longitudinally extending axis 15 and the transversely extending axis 17. In the specific embodiment shown in FIGS. 1 and 2 the non-printed portions 36a, 36b and 36c are substantially oval in shape and are structured and arranged such that they are spaced inwardly relative to the substantially transversely extending peripheral edge 40, the second opposed substantially transversely extending peripheral edge 42, the first longitudinally extending peripheral edge 44 and the opposed second longitudinally extending peripheral edge 46

As shown in FIG. 1, the first 36a, second 36b and third 36c non-printed portions of the longitudinally extending non-printed portions 36 are completely surrounded by the peripheral boundary 32 of the printed portion 24. In other words, the first 36a, second 36b and third 36c non-printed portions of the longitudinally extending non-printed portions 36 are contained entirely within the printed portion 24.

Each of the first 36a, second 36b and third 36c non-printed portions of the longitudinally extending non-printed portions 36 preferably have a width in the range of about 4.5 mm to about 7 mm and a length in the range of about 39 mm to about 99 mm, as measured along the path of such portion. As shown in FIG. 1, the second 36b and third 36 non-printed portions are preferably arranged such that they have a concave arrangement with respect to the longitudinally extending axis 15.

Referring to FIG. 2, the non-printed portions 22 further include a plurality of arcuate non-printed portions 38. Each of the arcuate non-printed portions 38 extend across the printed portion 24. Each of the arcuate non-printed portions 38 originates at one of the first substantially transversely extending peripheral edge 40, the second substantially transversely extending peripheral edge 42, the first longitudinally extending peripheral edge 44 and the opposed second longitudinally extending peripheral edge 46. Each of the arcuate non-printed portions 38 terminates at one of the first substantially transversely extending peripheral edge 40, the second substantially transversely extending peripheral edge 42, the first longitudinally extending peripheral edge 44 and the second longitudinally extending peripheral edge 46.

Each of the arcuate non-printed portions 38 preferably has a width in the range of about 2.5 mm to about 3.5 mm and a length in the range of about 24.3 mm to about 90.7 mm as measured along the path of such portion. As shown in FIG. 2, selected ones 38a of the non-printed portions 38 may be discontinuous. “Discontinuous” as used herein means that the non-printed portion 38 is formed by a plurality of discrete non-printed dots 41 or other discrete segments that are separated from an adjacent non-printed dot 41 by the printed portion 24.

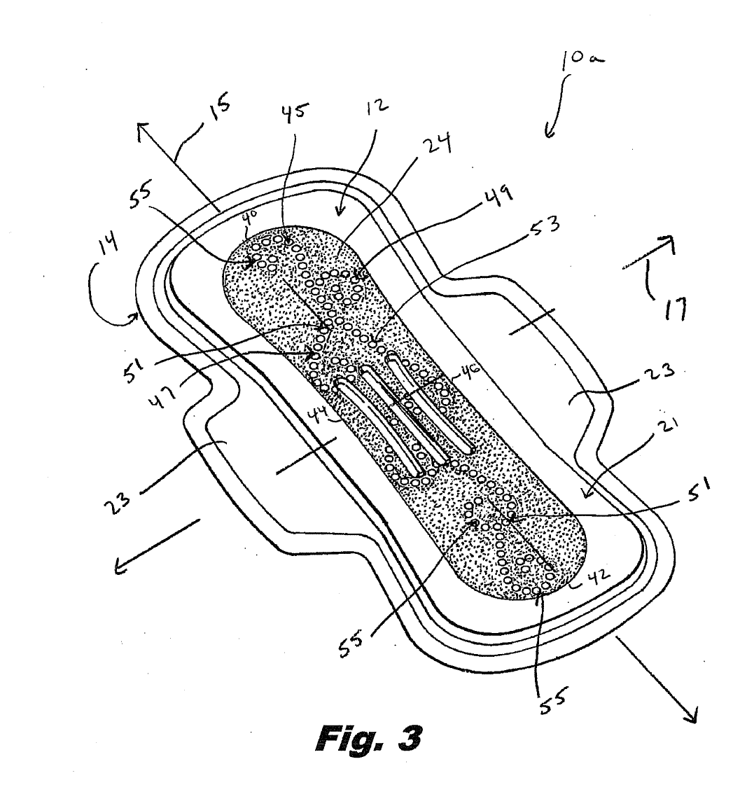

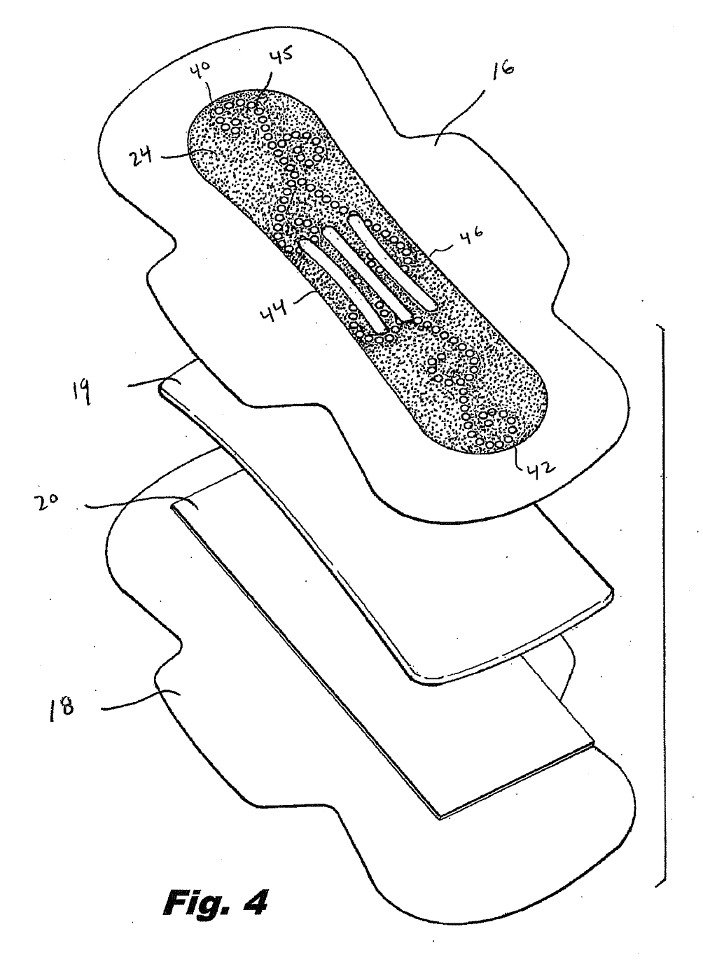

FIG. 3 provides a perspective view of a sanitary napkin 10a in accordance with a second embodiment of the present invention. The sanitary napkin 10a is similar to the sanitary napkin 10 described above with reference to FIG. 1. However, the sanitary napkin 10a shown in FIG. 3 does not include the plurality of arcuate non-printed portions 38. Rather, the sanitary napkin 10a includes an arcuate substantially longitudinally extending non-printed portion 45. As shown in FIG. 4, the arcuate substantially longitudinally extending non-printed portion 45 extends substantially from the first substantially transversely extending peripheral edge 40 to the second substantially transversely extending peripheral edge 42 of the printed portion 24.

As shown in FIG. 3 the arcuate substantially longitudinally extending non-printed portion 45 includes portions 47 that are located on a first side of the longitudinally extending axis 15 and portions 49 that are located on the opposite second side of the longitudinally extending axis 15. Thus, the arcuate substantially longitudinally extending non-printed portion 45 includes a plurality of portions 51 that cross over the longitudinally extending axis 15.

In the specific embodiment of the invention shown in FIG. 3, the arcuate substantially longitudinally extending non-printed portion 45 includes a central non-printed stem portion 53 and a plurality of concentric spiral portions 55 that extend from the stem portion 53. In the specific embodiment shown in FIG. 3 the arcuate substantially longitudinally extending non-printed portion 45 is “discontinuous”, however it may also be configured to be continuous in nature. Preferably, the arcuate substantially longitudinally extending non-printed portion 45 has a width in the range of about 2.5 mm to about 3.5 mm and a length in the range of from about 160 mm to about 190 mm as measured along a path of the portion 45.

In the embodiment shown in FIG. 3, the printed portion 24 preferably extends over a surface area of between about 28.3% and 32.8% of a total surface area of the top surface 12 of the main body 21 (i.e. excluding the area of the top surface 12 of the wings 23). Preferably, the printed portion 24 extends over an area of between about 5916 mm² and 6860 mm².

The printed portion 24 may be delivered to the sanitary napkins 10 and 10a described above by any one of or combination of the following: (1) color provided on the top surface 12 (i.e. body facing surface) of the cover layer 16; (2) color provided on the transfer layer 19 or core 20; (3) color provided on an insert layer arranged between the cover layer 16 and the transfer layer 19 or core 20 (if the transfer layer is omitted). In one embodiment of the invention the insert layer is a printed tissue layer. Of course, if the printed portion 24 is delivered by way of printing any layer located below the cover, the cover should be selected such that the printed portion 24 is visible from the top surface 12 of the article.

The printed portion 24 may be provided by any known means in the art including ink jet printing, flexographic printing or any other suitable printing technique known to those of skill in the art.

### Cover Layer

The cover layer 16 may be a relatively low density, bulky, high-loft non-woven web material. The cover layer 16 may be composed of only one type of fiber, such as polyester or polypropylene or it may include a mixture of more than one fiber. The cover 16 may be composed of bi-component or conjugate fibers having a low melting point component and a high melting point component. The fibers may be selected from a variety of natural and synthetic materials such as nylon, polyester, rayon (in combination with other fibers), cotton, acrylic fiber and the like and combinations thereof. Preferably, the cover layer 16 has a basis weight in the range of about 10 gsm to about 75 gsm.

Bi-component fibers may be made up of a polyester layer and a polyethylene sheath. The use of appropriate bi-component materials results in a fusible non-woven fabric. Examples of such fusible fabrics are described in U.S. Pat. No. 4,555,430 issued Nov. 26, 1985 to Chicopee. Using a fusible fabric increases the ease with which the cover layer may be mounted to the absorbent layers of the article and/or to the barrier layer.

The cover layer 16 preferably has a relatively high degree of wettability, although the individual fibers comprising the cover may not be particularly hydrophilic. The cover material should also contain a great number of relatively large pores. This is because the cover layer 16 is intended to take-up body fluid rapidly and transport it away from the body and the point of deposition. Therefore, the cover layer contributes little to the time taken for the napkin to absorb a given quantity of liquid (penetration time).

Advantageously, the fibers which make up the cover layer 16 should not lose their physical properties when they are wetted, in other words they should not collapse or lose their resiliency when subjected to water or body fluid. The cover layer 16 may be treated to allow fluid to pass through it readily. The cover layer 16 also functions to transfer the fluid quickly to the underlying layers of the napkin. Thus, the cover layer 16 is advantageously wettable, hydrophilic and porous. When composed of synthetic hydrophobic fibers such as polyester or bi-component fibers, the cover layer 16 may be treated with a surfactant to impart the desired degree of wettability.

Alternatively, the cover layer 16 can be made of a polymer film having large pores. Because of such high porosity, the film accomplishes the function of quickly transferring body fluid to the underlying absorbent layers.

The cover layer 16 may be attached to the underlying absorbent core 20, transfer layer 19, and/or the barrier layer 18, by adhesion and/or other suitable means know to those of skill in the art.

### Absorbent Core

In one embodiment, the absorbent core 20 is a blend or mixture of cellulosic fibers and superabsorbent disposed therein. Cellulosic fibers that can be used in the absorbent core 20 are well known in the art and include wood pulp, cotton, flax and peat moss. Wood pulp is preferred. Pulps can be obtained from mechanical or chemi-mechanical, sulfite, kraft, pulping reject materials, organic solvent pulps, etc. Both softwood and hardwood species are useful. Softwood pulps are preferred. It is not necessary to treat cellulosic fibers with chemical debonding agents, cross-linking agents and the like for use in the present material. Some portion of the pulp may be chemically treated as discussed in U.S. Pat. No. 5,916,670 to improved flexibility of the product. Flexibility of the material may also be improved by mechanically working the material or tenderizing the material.

The absorbent core 20 can contain any superabsorbent polymer (SAP) which are well known in the art. For the purposes of the present invention, the term “superabsorbent polymer” (or “SAP”) refers to materials which are capable of absorbing and retaining at least about 10 times their weight in body fluids under a 0.5 psi pressure. The superabsorbent polymer particles of the invention may be inorganic or organic crosslinked hydrophilic polymers, such as polyvinyl alcohols, polyethylene oxides, crosslinked starches, guar gum, xanthan gum, and the like. The particles may be in the form of a powder, grains, granules, or fibers. Preferred superabsorbent polymer particles for use in the present invention are crosslinked polyacrylates, such as the product offered by Sumitomo Seika Chemicals Co., Ltd. Of Osaka, Japan, under the designation of SA70N and products offered by Stockhausen Inc. In a specific example, the absorbent core is a material containing from 95% to about 40% percent cellulosic fiber by weight, and about 5% to about 60% SAP by weight.

### Transfer Layer

Adjacent to cover layer 16 layer on its inner side is the transfer layer 19. The transfer layer 19 provides the means of receiving body fluid from the cover layer 16 and holding it until the absorbent core 20 has an opportunity to absorb the fluid, and therefore serves as a fluid transfer or acquisition layer. In addition the transfer layer 19 functions to wick the fluid in the longitudinal and transverse directions of the napkin so that the full absorbent capacity of the napkin is utilized.

The transfer layer 19 preferably has a larger proportion of smaller pores than the cover layer 16. These attributes allow the transfer layer 19 to contain body fluid and hold it away from the outer side of the cover layer 16, thereby preventing the fluid from re-wetting the cover layer 16 and its surface.

The transfer layer 19 may be composed of fibrous materials, such as wood pulp, polyester, rayon, flexible foam, or the like, or combinations thereof. The transfer layer 19 may also comprise thermoplastic fibers for the purpose of stabilizing the layer and maintaining its structural integrity. The transfer layer 19 may be treated with surfactant on one or both sides in order to increase its wettability, although generally the transfer layer 19 is relatively hydrophilic and may not require treatment. The transfer layer 19 is preferably bonded on both sides to the adjacent layers, i.e. the absorbent core 20 and the barrier layer 18.

### Barrier Layer

Underlying the transfer layer 19 is a barrier layer 18 comprising liquid-impervious film material so as to prevent liquid from egressing the sanitary napkin and staining the wearer's undergarment. The barrier layer 18 is preferably made of polymeric film, although it may be made of liquid impervious, air-permeable material such as repellent-treated non-woven or micropore films or foams.

The barrier layer 18 may be breathable, i.e., permits vapor to transpire. Known materials for this purpose include nonwoven materials and microporous films in which microporosity is created by, inter alia, stretching an oriented film. Single or multiple layers of permeable films, fabrics, melt-blown materials, and combinations thereof that provide a tortuous path, and/or whose surface characteristics provide a liquid surface repellent to the penetration of liquids may also be used to provide a breathable backsheet. The cover layer 16 and the barrier layer 18 are preferably joined along their marginal portions so as to form an enclosure or flange seal that maintains the transfer layer 19 and absorbent core 20 captive. The joint may be made by means of adhesives, heat-bonding, ultrasonic bonding, radio frequency sealing, mechanical crimping, and the like and combinations thereof.

Positioning adhesive may be applied to the bottom surface 14 of the barrier layer 18 for securing the napkin 10 to a garment during use. The positioning adhesive may be covered with removable release paper so that the positioning adhesive is protected by the removable release paper prior to use.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of the invention.

We claim:

1. An absorbent article comprising:
a longitudinally extending axis;
a transversely extending axis;
a top surface;
a cover layer;
a barrier layer;
an absorbent core arranged between the cover layer and the barrier layer;
a printed portion; and
a plurality of non-printed portions;
wherein the printed portion and the plurality of non-printed portions are arranged to create a perception of a functional benefit by a user looking upon the top surface of the absorbent article.

2. The absorbent article according to claim 1, wherein the functional benefit includes at least one of breathability of the absorbent article, dryness, temperature control, body fit, comfort, and combinations thereof.

3. The absorbent article according to claim 2, wherein the printed portion is centrally arranged on a main body of the absorbent article and extends substantially in a longitudinal direction of the absorbent article.

4. The absorbent article according to claim 3, wherein the printed portion is substantially peanut shaped.

5. The absorbent article according to claim 3, wherein printed portion extends over a surface area of between about 24% and about 33% of a total surface area of the top surface of the main body.

6. The absorbent article according to claim 3, wherein an entirety of the printed portion has a single color.

7. The absorbent article according to claim 5, wherein the printed portion extends over a surface area of between about 5203 mm² and about 6860 mm².

8. The absorbent article according to claim 7, wherein the printed portion is arranged such that it is symmetrically arranged with respect to the longitudinally extending axis and the transversely extending axis.

9. The absorbent article according to claim 1, wherein the plurality of non-printed portions includes a first non-printed portion that that completely surrounds the printed portion.

10. The absorbent article according to claim 9, wherein the first non-printed portion extends from a peripheral boundary of the printed portion to a peripheral edge of the main body.

11. The absorbent article according to claim 10, wherein the plurality of non-printed portions further includes a plurality of substantially longitudinally extending non-printed portions that are substantially centrally arranged within the printed portion.

12. The absorbent article according to claim 11, wherein the a plurality of substantially longitudinally extending non-printed portions includes a first central non-printed portion that is symmetrically arranged with respect to the longitudinally extending axis and the transversely extending axis.

13. The absorbent article according to claim 12, wherein the a plurality of substantially longitudinally extending non-printed portions includes a second and third non-printed portion that are arranged in spaced relationship to the first central non-printed portion and are located on opposite sides of the longitudinally extending axis.

14. The absorbent article according to claim 13, wherein the second and third non-printed portion are symmetrically arranged with respect to the transversely extending axis.

15. The absorbent article according to claim 14, wherein the first, second and third non-printed portion are completed surrounded by the peripheral boundary of the printed portion.

16. The absorbent article according to claim 13, wherein the plurality of non-printed portions further includes a plurality of arcuate non-printed portions.

17. The absorbent article according to claim 16, wherein the printed portion includes a first substantially transversely extending peripheral edge, a second opposed substantially transversely extending peripheral edge, and first and second opposed longitudinally extending peripheral edges.

18. The absorbent article according to claim 17, wherein each one of the plurality of arcuate non-printed portions originates at one of (i) the first substantially transversely extending peripheral edge, (ii) the second opposed substantially transversely extending peripheral edge, (iii) the first longitudinally peripheral edge, and (iv) the second longitudinally extending peripheral edge.

19. The absorbent article according to claim 18, wherein each one of the plurality of arcuate non-printed portions terminates at one of (i) the first substantially transversely extending peripheral edge, (ii) the second opposed substantially transversely extending peripheral edge, (iii) the first longitudinally peripheral edge, and (iv) the second longitudinally extending peripheral edge.

20. The absorbent article according to claim 19, wherein selected ones of the plurality of arcuate non-printed portions are continuous and selected ones of the plurality of arcuate non-printed portions are discontinuous.

21. The absorbent article according to claim 11, wherein the plurality of non-printed portions includes an arcuate substantially longitudinally extending non-printed portion.

22. The absorbent article according to claim 21, wherein the printed portion includes a first substantially transversely extending peripheral edge, a second opposed substantially transversely extending peripheral edge, and first and second opposed longitudinally extending peripheral edges.

23. The absorbent article according to claim 22, wherein the arcuate substantially longitudinally extending non-printed portion extends substantially from the first substantially transversely extending peripheral edge to the second opposed substantially transversely extending peripheral edge.

24. The absorbent article according to claim 23, wherein the arcuate substantially longitudinally extending non-printed portion includes portions that are located on a first side of the longitudinally extending axis and portions that are located on the second side of the longitudinally extending axis.

25. The absorbent article according to claim 24, wherein the arcuate substantially longitudinally extending non-printed portion includes a plurality of portions that cross over the longitudinally extending axis.
